(19) United States
(12) Patent Application Publication
(10) Pub. No.: US 2021/0002727 A1
(43) Pub. Date: Jan. 7, 2021

(54) METHOD OF DETERMINING DISEASE-ASSOCIATED GENE VARIANTS AND ITS USE IN THE DIAGNOSIS OF LIVER CANCER AND FOR DRUG DISCOVERY

(71) Applicant: Globe Biotech Ltd., Dhaka (BD)

(72) Inventors: Naznin Sultana, Dhaka (BD); Mijanur Rahman, Dhaka (BD); Sanat Myti, Dhaka (BD); Md Jikrul Islam, Dhaka (BD); Md Golam Mustafa, Dhaka (BD); Kakon Nag, Dhaka (BD)

(21) Appl. No.: 16/914,706

(22) Filed: Jun. 29, 2020

Related U.S. Application Data
(60) Provisional application No. 62/869,362, filed on Jul. 1, 2019.

(51) Int. Cl. C12Q 1/6886

(52) U.S. Cl. CPC C12Q 1/6886; A61K 45/06

(57) ABSTRACT

A method of identifying disease-associated gene variants in a patient is provided. The method includes conducting exome sequencing of a nucleic acid-containing sample from the patient identify nucleic acid variants within the sample; filtering out non-disease-related nucleic acid variants by comparison with known sequence variants from the specific ethnic background of the patient, somatic mutations and common non-disease-related sequence variants; and conducting a comparison of the filtered sequence against a healthy control nucleic acid sequence to identify disease-associated sequence variants. Novel gene variants associated with liver cancer identified using the method are also provided.

### CROSS-REFERENCE TO RELATED APPLICATIONS

This application is a non-provisional patent application of and claims priority to U.S. Provisional Application No. 62/869,362 filed Jul. 1, 2019, which is incorporated herein by reference in its entirety.

### FIELD

The present invention generally relates to a method of identifying disease-associated gene variants, and to its use in the diagnosis of disease, for example, diagnosis of cancer, and for drug discovery.

### BACKGROUND

Cancer is a multifactorial disease mostly influenced by genetics and environmental factors. At the genetic level, a cancerous phenomenon results from the accumulation of genomic alterations leading to the dysregulation of cell proliferation, regeneration and apoptosis. Hepatocellular carcinoma (HCC) is the fifth most common human cancer among different types of cancer, with approximately 750,000 new cases occurring worldwide each year. About 85% of hepatocellular carcinoma (HCC) patients are from developing countries, such as Southeast Asia and sub-Saharan Africa, and worldwide death from liver cancer is 50%.

Treatment strategies for patients with HCC include surgery, radiation, chemotherapy, liver transplantation, and targeted therapies. Although there have been improvements in the diagnosis and treatment protocols, the death rates are increasing for patients with HCC. The majority of studies showed that 5-year survival rate is less than 5%.

Next generation sequencing (NGS) has been advancing the progress of detection of disease-associated genetic variants and genome-wide profiling of expressed sequences over the past decade. NGS enables the analyses of multiple regions of a genome in a single reaction format and has been shown to be a cost-effective and efficient tool for root-cause analysis of disease and optimization of treatment. NGS has been leading global efforts to devise personalized and precision medicine (PM) in clinical practice. Despite the effectiveness of NGS for detection of disease-associated genetic variants, definitive prediction of cancer markers for all types of diseases and for global populations remains challenging due to the diversity of cancer types and genetic variants in humans.

Cancer associated genomic alterations are generally global as opposed to local in nature. Gross chromosomal structure alterations by amplification, deletion, translocation and/or inversion of chromosomal segments are considered as common characteristics of cancer genomes. The heterogenous nature of cancers on both a spatial and temporal scale has diversified the cancerous genome at the individual level. A significant number of studies relating to liver cancer indicate that NGS plays a valuable role in cancer diagnosis, classification and treatment. Importantly, a comprehensive assessment of cancer genome-associated genetic alteration plays an important role in predicting oncology drugs and therapeutic outcomes. However, one of the challenges associated with NGS is the analysis and subsequent extraction of meaningful information from an overwhelming amount of data that is generated by NGS.

In view of the impact of cancer, it would be desirable to develop a novel method of diagnosing cancer, and in particular, HCC.

### SUMMARY

A novel method has been developed which permits the identification of disease-related genetic variants. In one embodiment, genetic variants associated with liver cancer have been identified.

In one aspect of the invention, a method of determining disease-associated gene variants in a patient of a specific ethnic background having a disease is provided comprising the steps of:

i) obtaining a nucleic acid sample from a patient and conducting exome sequencing of the sample to identify nucleic acid variants within the sample;

ii) filtering out non-disease-related nucleic acid variants by comparison with known sequence variants from the specific ethnic background, somatic mutations and common non-disease-related sequence variants; and

iii) conducting a comparison of filtered sequence against a healthy control nucleic acid sequence to identify disease-associated sequence variants.

In another aspect of the invention, a method of identifying a gene variant of at least one of KRT6A, MUC16, PRKCG, TRIOBP, RELN, NUDT18, MAP1S, SNX27, AUP1, MIR5004, SVEP1, SORD, VPS33B, MRPL38, AP5B or MYH6, or a protein it encodes, in a patient sample is provided. The method comprises the steps of:

i) contacting a biological sample obtained from the patient with a reactant that binds to at least one of the gene variants or protein it encodes; and

ii) detecting the presence of the gene variant or protein in the sample by detecting binding of the reactant with the gene variant or protein.

In another aspect of the invention, a method of diagnosing liver cancer in a patient is provided comprising the steps of:

i) contacting a biological sample obtained from the patient with a reactant that binds to at least one gene variant of KRT6A, MUC16, PRKCG, TRIOBP, RELN, NUDT18, MAP1S, SNX27, AUP1, MIR5004, SVEP1, SORD, VPS33B, MRPL38, AP5B or MYH6, or protein it encodes;

ii) detecting the presence of at least one of the gene variants or proteins in the sample by detecting binding of the reactant with the gene variant or protein; and

iii) diagnosing the patient with liver cancer when the presence of the gene variant is detected.

These and other aspects of the invention are described herein by reference to the following figures.

### BRIEF DESCRIPTION OF THE FIGURES

FIG. 1 illustrates filter chains applied for variant detection including: (A) SNP detection filter chain; (B) MNV detection filter chain; (C) CNV detection filter chain; and (D) INDEL detection filter chain;

FIG. 2 illustrates the whole-exome sequencing (WES) landscape constructed with Ion Reporter™ Genomic Viewer (IRGV);

FIG. 3 is a heat map of SNP variant impacts. (A) SNP with frameshift Insertion (SIFT score: 0.00-1), and (B) SNP with frameshift deletion (SIFT score: 0.00-1).

FIG. 4 is a heat map of MNV variant impacts. (A) MNV with frameshift Insertion (SIFT score: 0.00-1). (B) SNP with nonsense mutation (SIFT score: 0.00-1).

FIG. 5 is a heat map of INDEL variant impacts. (A) INDEL with frameshift insertion (SIFT score: 0.00-1). (B) INDEL with nonsense mutation (SIFT score: 0.00-1). (C) IRGV visualization for locus chr19:14829263 of GBNGS011.

FIG. 6 illustrates a tissue specific expression profile of 17 neoplasm exclusive genes in liver.

### DETAILED DESCRIPTION

A method of determining disease-associated gene variants in a patient from a specific population having a disease. The method comprises the steps of: i) obtaining a nucleic acid sample from a patient and conducting exome sequencing of the sample to identify nucleic acid variants within the sample; ii) filtering out non-disease-related nucleic acid variants by comparison with known sequence variants from the specific population, somatic mutations and common non-disease-related sequence variants; and iii) conducting a comparison of filtered sequence against a healthy control nucleic acid sequence to identify disease-associated sequence variants.

The nucleic acid sample of a patient may be obtained from a biological sample, including whole blood, plasma, serum, or saliva, urine, cerebrospinal fluid or other bodily fluids, or tissue samples, e.g. buccal, urinary or other tissue, obtained from the patient. The sample may be obtained using methods well-established in the art, and may be obtained directly from the patient or may be obtained from a sample previously acquired from the patient which has been appropriately stored for future use (e.g. stored at 4° C.). Nucleic acid is then extracted from the sample using well-established methods. Proteins may also be extracted from the biological sample to provide a protein sample from the patient for analysis. An amount of sample of at least about 100 μl, e.g. 100 μl of diluted human serum (1:100 dilution in blocking buffer) may be used to conduct the present method. The term “patient” is used herein to refer to both human and non-human mammals including, but not limited to, cats, dogs, horses, cattle, goats, sheep, pigs and the like.

Once obtained, the sample is subjected to exome sequencing, also known as whole exome sequencing (WES). WES is a genomic technique for sequencing all of the protein-coding region of genes in a genome (known as the exome). It consists of two steps: the first step is to select only the subset of DNA that encodes proteins. These regions are known as exons—humans have about 180,000 exons, constituting about 1% of the human genome, or approximately 30 million base pairs. The second step is to sequence the exonic DNA using any high-throughput DNA sequencing technology, e.g. sequencing techniques including methods which apply “sequencing by synthesis” such as pyrosequencing and Sequenom® analysis, and other Next Generation Sequencing (NGS) methods (e.g. such as Illumina, 454, Ion torrent and Ion proton sequencing).

Following sequencing, the nucleic acid sample is filtered to remove non-disease-related nucleic acid variants such as nucleic acid variants common to the specific population, such as the ethnically-related sequence variants, somatic mutations and other common non-disease-related sequence variants such as single and multiple nucleotide variants, copy number variants, and insertion or deletion polymorphism variants. Such non-disease-related nucleic acid variants are identified by comparison of the sample against healthy control sequences. Sequence variants common to both the sample and the controls may then be excluded.

Nucleic acid variants are then identified that exist within the sample nucleic acid only and not in the healthy control sequences. The gene or genes containing the identified nucleic acid variants are identified as disease-related genes.

The present method may be used to identify gene variants associated with any disease, including but not limited to, Alzheimer's disease, Crohn's disease, type 2 diabetes, Parkinson's Disease, Muscular Dystrophy, Hemophilia A, Glucose-Galactose (Malabsorption Syndrome), Amyotrophic Lateral Sclerosis, ADA Immune Deficiency, Familial Hypercholesterolemia, Myotonic Dystrophy, Amyloidosis, Neurofibromatosis, Cancer, Polycystic Kidney Disease, Tay-Sachs Disease, Retinoblastoma, Phenylketonuria, Sickle-Cell Anemia, Multiple Endocrine Neoplasia, Type 2, Melanoma, Werner Syndrome, Cystic Fibrosis, Spinocerebellar Ataxia, Hemochromatosis, Familial Adenomatous (FAP), Huntington Disease, Retinitis Pigmentosa, Ehlers-Danlos syndrome, Gaucher Disease, Azoospermia, Adrenoleukodystrophy, auto immune disease, rheumatic arthritis etc.

In one embodiment, the method is used to identify gene variants associated with a cancer, for example, carcinoma such as bladder, breast, colon, kidney, brain, liver, lung, including small cell lung cancer, esophagus, gall-bladder, ovary, pancreas, stomach, cervix, thyroid, prostate, and skin, including squamous cell carcinoma; sarcomas; malignant neoplasms; hematopoietic tumours of lymphoid lineage including leukaemia, acute lymphocytic leukaemia, acute lymphoblastic leukaemia, B-cell lymphoma, T-cell-lymphoma, Hodgkin's lymphoma, non-Hodgkin's lymphoma, hairy cell lymphoma and Burkitt's lymphoma; hematopoietic tumours of myeloid lineage including acute and chronic myelogenous leukemias, myelodysplastic syndrome and promyelocytic leukaemia; tumours of mesenchymal origin, including fibrosarcoma and rhabdomyosarcoma; tumours of the central and peripheral nervous system, including astrocytoma neuroblastoma, glioma and schwannomas; other tumours, including melanoma, seminoma, teratocarcinoma, osteosarcoma, xeroderma pigmentosum, keratoxanthoma, thyroid follicular cancer, Kaposi's sarcoma and pediatric cancers from embryonal and other origins.

Use of the present method has revealed novel gene variants associated with liver cancer, namely gene variants within the genes, KRT6A, MUC16, PRKCG, TRIOBP, RELN, NUDT18, MAP1S, SNX27, AUP1, MIR5004, SVEP1, SORD, VPS33B, MRPL38, AP5B1, and MYH6. Of these genes, SVEP1 (EGF and Pentraxin Domain Containing 1); SORD (Sorbitol Dehydrogenase); MRPL38 (Mitochondrial Ribosomal Protein L38); and KRT6A (Keratin 6A) exhibited the greatest protein expression.

Thus, in another aspect of the invention a method of identifying a gene variant of at least one of SVEP1, SORD, MRPL38 and KRT6A, or the protein it encodes, in a patient is provided. The method comprises contacting a biological sample obtained from the patient with a reactant that specifically binds to at least one of the gene variants or protein it encodes; and detecting the presence of the gene variant or protein it encodes in the sample by detecting binding of the reactant with the gene variant or protein it encodes. The reactant may be any reactant that specifically binds to the target gene variant or protein it encodes.

In one embodiment, the gene variants associated with liver cancer are as follows: for SORD, a mutation of the gene to replace T at position 416 of the gene with C (which results in a change at amino acid position 139 of the protein from Phe to Ser); for KRT6A, GC at position 1048-1049 of the gene is replaced by CG (which results in a change at amino acid position 350 from Ala to Arg); for SVEP1, a mutation of the gene to replace G at position 1159 of the gene with T (which results in a change at amino acid position 387 of Gly to Cys); and for MRPL38, a mutation of the gene to replace G at position 430 of the gene with C (which results in a change at amino acid position 144 of Gly to Arg).

The full sequences of the genes referred to herein are known in the art and may be obtained by reference to publicly available databases such as the NCBI (National Center for Biotechnology Information). For example, the transcript sequence for SVEP1 and the protein it encodes are provided by NCBI accession nos. NM_153366.4 and NP_699197.3, respectively; the transcript sequence for SORD and the protein it encodes are provided by NCBI accession nos. NM_003104.6 and NP_003095.2, respectively; the transcript sequence for MRPL38 and the protein it encodes are provided by NCBI accession nos. NM_032478.4 and NP_115867.2, respectively; and the transcript sequence for KRT6A and the protein it encodes are provided by NCBI accession nos. NM_005554.4 and NP_005545.1, respectively.

The reactant may be an oligonucleotide reactant that specifically binds to the target gene variant, for example, an oligonucleotide probe that is complementary to the target gene variant and specifically hybridizes thereto. Oligonucleotide probes are readily designed based on the sequences of the target gene variant, as denoted above, based on the known transcript sequences of the endogenous target genes available on sequence databases such as NCBI and Uniprot. Oligonucleotide probes are generally labelled, for example with radioisotopes, epitopes, biotin/streptavidin, or fluorophores to enable their detection.

Alternatively, the reactant may be an anti-double stranded DNA (anti-dsDNA) antibody that binds to a target gene variant. Enzyme-linked immunosorbent assay (ELISA) or immunofluorescent labels may be used to detect binding of the antibody to the target gene variant.

To identify a protein encoded by a gene variant, an antibody that binds to the protein encoded by the target gene variant may be used and binding may be identified, for example, by ELISA or immunofluorescence.

In a further embodiment, nucleic acid (DNA or RNA) is isolated and purified from a sample obtained from a patient (either from blood cells or urinary cells or buccal cells or any other tissue sample). The target genes are amplified using gene-specific PCR (polymerase chain reaction). The presence of one or more of the target gene variants is identified by sequencing (forward and reverse direction sequencing), using any one of a number of nucleic acid sequencing techniques.

The identification of gene or protein variants associated with disease facilitates drug discovery. For example, gene therapies may be developed based on the detection of gene variants associated with a given disease. Such therapies may be designed to introduce a normal gene or genes that function to express a necessary protein that is no longer appropriately expressed by the gene variant because it is either faulty or not expressed at all. Gene therapies including CRISPR technologies may also be used to edit or correct gene variants such that normal protein expression is resumed. Protein replacement therapies may also be developed if it is determined that gene variants associated with a disease express non-functioning proteins, or fail to express a required protein.

In a further aspect, a method of diagnosing liver cancer in a patient is provided. The method comprises contacting a biological sample obtained from the patient with a reactant that specifically binds to at least one gene variant of KRT6A, MUC16, PRKCG, TRIOBP, RELN, NUDT18, MAP1S, SNX27, AUP1, MIR5004, SVEP1, SORD, VPS33B, MRPL38, AP5B or MYH6, or a protein it encodes. The presence of at least one of the gene variants or protein it encodes is detected by detecting binding of the reactant with the gene variant or protein it encodes. The patient is diagnosed with liver cancer when the presence of the gene variant, or protein it encodes, is detected.

A patient diagnosed with liver cancer based on the presence of the gene variants of SVEP1, SORD, MRPL38 and/or KRT6A, or the proteins they encode, may be treated using an appropriate treatment, for example, chemotherapy, surgery and/or radiation. Other treatments that may be used include cell therapy, gene therapy, siRNA treatment, shRNA treatment, therapeutic antibodies, gene-function check point modulators and molecular targeted cancer therapies.

Embodiments of the invention are described in the following specific examples which are not to be construed as limiting.

### Example 1

### METHODS and MATERIALS

Subject Selection—

NGS-based genomic landscape analysis was performed on four human South Asian subjects: one metastatic cancer patient (GBNGS011 subject) and three asymptomatic healthy subjects comprising two males and one female. The selected subjects were aged between 50-70 years old. Samples were collected following the institutional ethical policy. The clinical-pathologic features of the neoplasm of the liver patient included hepatomegaly with a large space occupying lesion (SOL) in the right lobe of the liver. Fine needle aspiration from the SOL of the liver showed numerous malignant cells with a finely granular chromatin pattern and hemorrhagic background under microscopy. Additionally, elevated Alpha-Feto protein level (207.0 IU/mL) was estimated in the blood.

Genomic DNA Isolation—

The analysis was performed on the genomic DNA extracted from the blood samples of the patients. An automated platform (MagMAX™ Express-96 Magnetic Particle Processor; Life Technologies, USA) was used to extract the genomic DNA from the blood samples following the manufacturer's instructions of MagMAX™ DNA Multi-Sample Kits (Life Technologies, USA). The quantity of the extracted DNA was estimated using Qubit™ dsDNA HS assay kit (Life Technologies, USA) in combination with Qubit™ fluorometer (Life Technologies, USA).

Next-Generation Sequencing (NGS)—

NGS was performed as previously described by Fujita (Biomed. Rep. 2017; 7:17-20) and Damiati (Hum. Genet. 2016; 135:499-511). In brief, 100 ng of DNA was amplified for genomic library preparation using the exome enrichment kit (Ion AmpliSeg™ Life Technologies, USA) in order to sequence the key exonic regions (>97% of Consensus Coding Sequences (CCDSs)) of the genome. Ion Chef™ System (Life Technologies, USA) was used for template preparation and enrichment using Ion540™ Kit—Chef (Life Technologies, USA). The same automated platform was used for loading Ion540™ Chips with template-positive Ion Sphere™ Particles. Exome sequencing was performed on the Ion S5™ XL Sequencer (Life Technologies, USA) with the loaded chips. Data analysis was done by Torrent Suite™ Software (v 5.2.2; Life Technologies, USA). Coverage analysis was performed using Coverage Analysis plug-in (v5.2.0.9). Variant Caller plug-in (v5.2.0.34) was used for mutations/variants detection against reference genome (hg19).

Data Filtering and Prioritization—

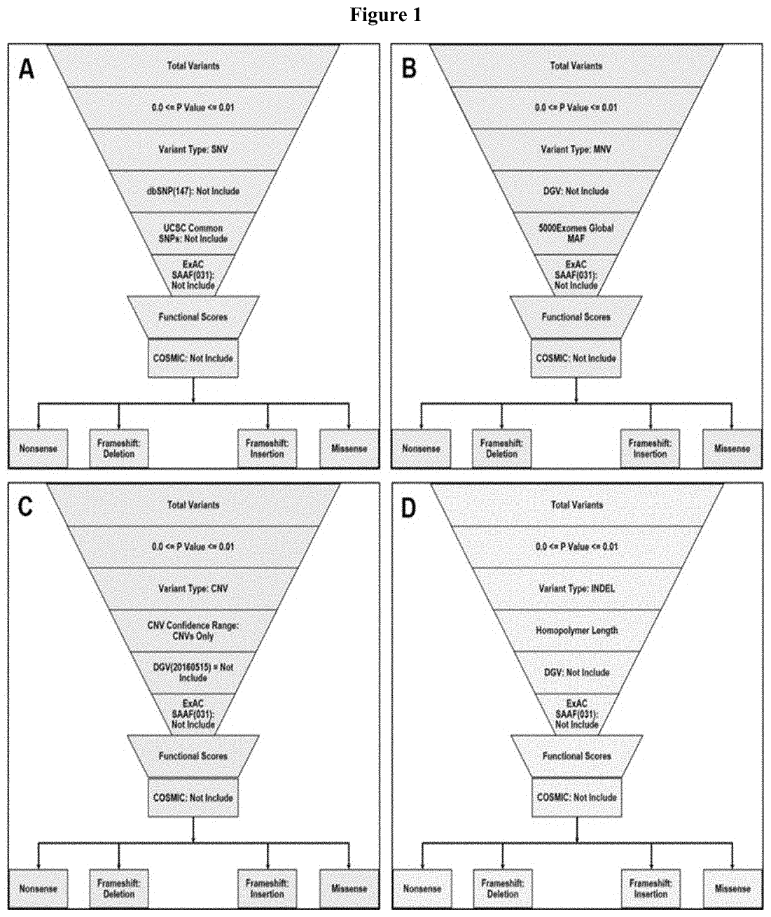

The Variant Call Format (VCF) and binary version of SAM (Sequenced Aligned Map) (BAM—Binary Aligned Map) files for all samples were uploaded into Ion Reporter™ 5.10.2.0 (Life Technologies, USA) for data filtering and prioritization using variant specific filter chain (FIG. 1) for identifying liver-cancer specific genetic variants. Total variants of each sample were detected by “Variant Caller” plug-in where the p-value was 0.0-0.01. Despite using numbers of bioinformatics data repositories, retrieved variants through most extensive and curated servers, categorized according to the variants type, and then imposing distinct variant-specific, customized filter chains. After that, “Exome Aggregation Consortium South Asian Allelic Frequency (ExAC SAAF)” hits were filtered out for elimination of rare genetic variation for “South Asian” population. Remaining variants were then filtered by worse functional impact (SIFT score: 0.0-0.05; PolyPhen score: 0.85-1.0) and deleterious evolutionary distance (Grantham scores: 101-215), respectively. Somatic mutations across the range of human cancers were excluded by applying “Catalogue of Somatic Mutations in Cancer (COSMIC)” filter. After filtering all common variants, existing variants were classified according to the variant effect (e.g., nonsense, missense, frameshift insertion and frameshift insertion mutations). For single-nucleotide variant (SNV) applying “Single Nucleotide Polymorphism Database (dbSNP)” and “UCSC common SNPs” database, for Multiple-nucleotide variant (MNV) applying “Database of Genomic Variants (DGV)” and “5000Exomes” database, for copy number variant (CNV) applying CNV confidence range and DGV database, and for insertion or deletion polymorphism (INDEL) applying homopolymer length filter and DGV database, as shown in FIG. 1. Variants matching with dbSNP, UCSC common SNPs, DGV and 5000Exomes database were excluded for downstream prioritization.

Data Selection for Exclusive Mutation—

With the variant pools obtained from database analyses, data were curated for finding intra-subject match hits at least 100× coverage. A variant was considered to be neoplasm-specific if and only if it occurred exclusively in the GBNGS011 subject. The hits were then screened for liver-specific protein expression profile, and spatial functional and biological significance through comparison of “GeneCards” entries.

### Results

Coverage Analysis and Variant Detection—

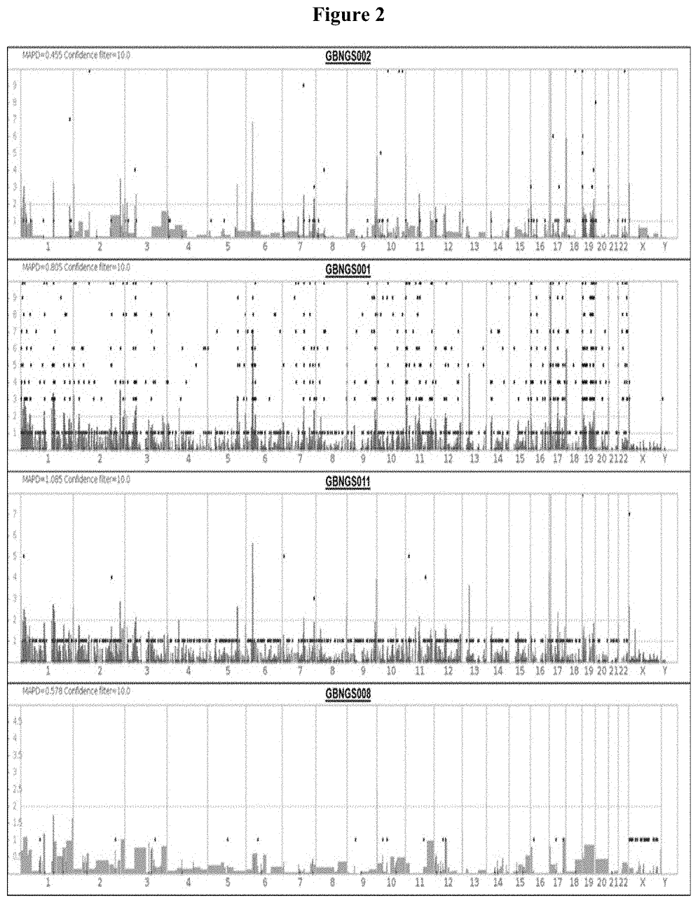

The whole-exome sequencing (WES) data from 4 subjects were aligned against the reference genome hg19 for the analysis of coverage and detection of variants (FIG. 2). The ‘x’-axis indicates chromosome number and ‘y’-axis indicates confidence filter for CNV. The results are shown in Table 1. The range of the mean depth of coverage was 30-233. The sequence from GBNGS011 has the lowest percentage of mapped read. GBNGS011 aligned on target with minimal variant (23197) calls and GBNGS002 aligned on target with maximum variant (39339) calls.

TABLE 1
Analysis of coverage and variants detection.

| Sample (accession no.) | On Target (%) | Mean Depth | Variants |
|---|---|---|---|
| GBNGS001 (SRR8293457) | 92.63 | 170.7 | 35635 |
| GBNGS002 (SRR8293456) | 96.85 | 233.4 | 39339 |
| GBNGS008 (SRR8293455) | 92.46 | 30.79 | 23197 |
| GBNGS011 (SRR8293454) | 90.88 | 42.79 | 25842 |

SNV Detection—

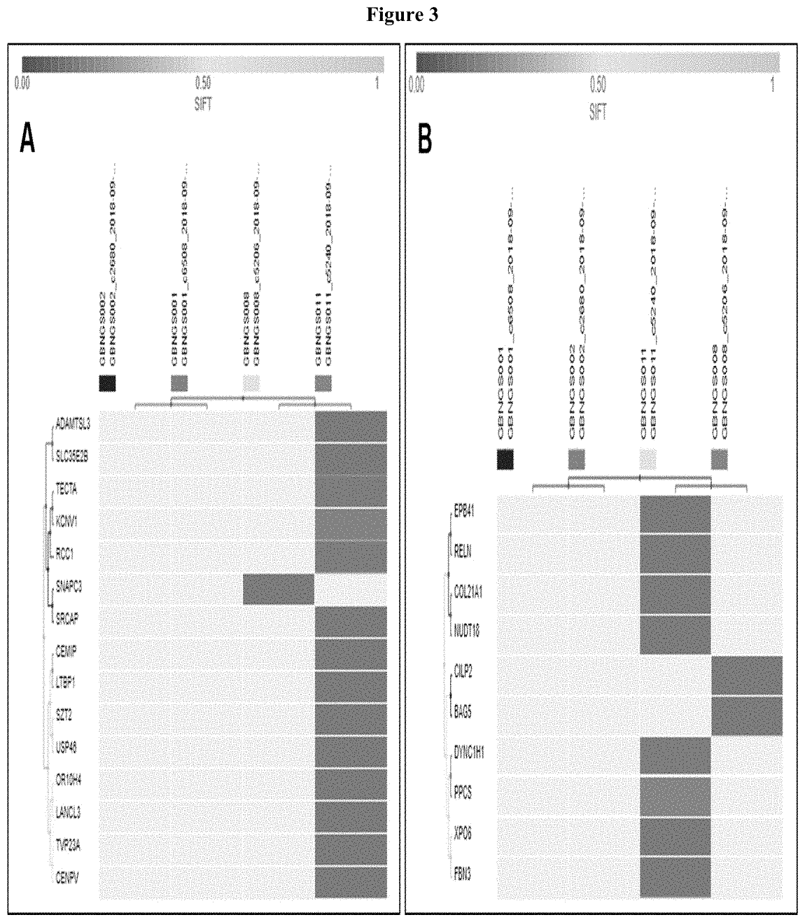

The exome data from the 4 subjects were filtered through SNP detection filter chain that consists of seven different filters (FIG. 1A). SNP detection filter chain filtered 411 SNVs from 121,556 variants associated with 400 genes. All the variants were recognized as missense mutation by default. Besides, frameshift deletion mutations were detected in 15 genes (CDK11B, RCC1, SZT2, LTBP1, USP46, KCNV1, TECTA, CEMIP, ADAMTSL3, TVP23A, SRCAP, CENPV, OR10H4, and LANCL3). GBNGS011 possesses almost all these mutated genes except SNAPC3 that was found only in GBNGS008 (FIG. 3A). Among these, 10 genes with SNV-associated frameshift insertion mutation were identified, including EPB41, PPCS, COL21A1, RELN, NUDT18, DYNC1H1, BAG5, XPO6, FBN3, and CILP2 (FIG. 3B). Among the study cohort, GBNGS008 carried mutations on CILP2 and BAG5 genes and, the rest of the mutations were carried only by GBNGS011. No nonsense mutation was detected by SNP detection filter chain.

MNV Detection—

MNV detection filter chain (FIG. 1B) generated 222 variants from 121,556 variants associated with 219 genes. 222 variants were recognized as missense mutations. MNV-associated frameshift insertion mutation filtering analysis revealed that except for the MEGF6 gene from GBNGS008, all other genes, viz., SLC30A1, EXTL3, FOXB2, FBXL14, NOC4L, CCDC78, MT4, IRF8, PRR14L, and TRIOBP were from GBNGS011 (FIG. 3A). Functional impact SIFT score ranges from 0.00 (which represents deleterious effect in genes) to 1 (which represents tolerated effect in genes). Variants with scores closer to 0.00 are more confidently predicted to be deleterious. Variants with scores 0.05 to 1 are predicted to be tolerated (benign). Horizontal axis represents the gene order distance.

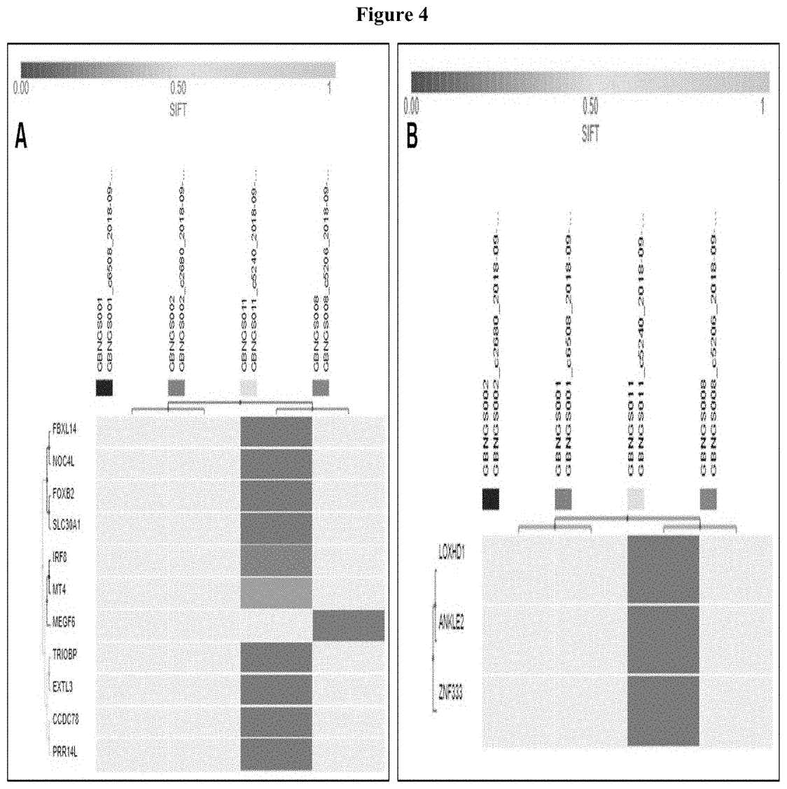

MNV-induced nonsense mutations were also observed in ZNF333, ANKLE2 and LOXHD1 genes (FIG. 4B) in GBNGS011. A total of 42 genes were found to be associated with frameshift deletion mutation due to MNV. Among these gene pools; GBNGS008, GBNGS001 and GBNGS002 carried mutations in KLHL5 gene, PIF1 gene and DDX60 gene, respectively. The rest of the mutations (on ATR, DNAH10, ARID4B, ADAMTS7, JMJD6, MAP3K6, KLK12, SF3A3, B4GALT3, HSD17B3, SCG5, PFAS, ARSD, NOS2, KCND2, CUBN, MUC2, WDCP, AHNAK2, SLC25A29, DNAH2, TJP3, MEPCE, PKD2, TXNDC11, GTF3A, MYO15A, PHF1, RBM4, RBM14-RBM4, ATP5J2-PTCD1, PTCD1, IFT46, NRCAM CHPF2, SH2B1, METTL23, SNN, and MTIF3 gene) were in GBNGS011. Functional impact SIFT score ranges from 0.00 (which represents deleterious effect in genes) to 1 (which represents tolerated effect in genes). Variants with scores closer to 0.00 are more confidently predicted to be deleterious. Variants with scores 0.05 to 1 are predicted to be tolerated (benign). Horizontal axis represents the gene order distance.

CNV Detection—

CNV detection filter chain (FIG. 1C) primarily filtered 85 CNVs from 121,556 variants. Applying the COSMIC filter ultimately nullified the CNV output.

INDEL Detection—

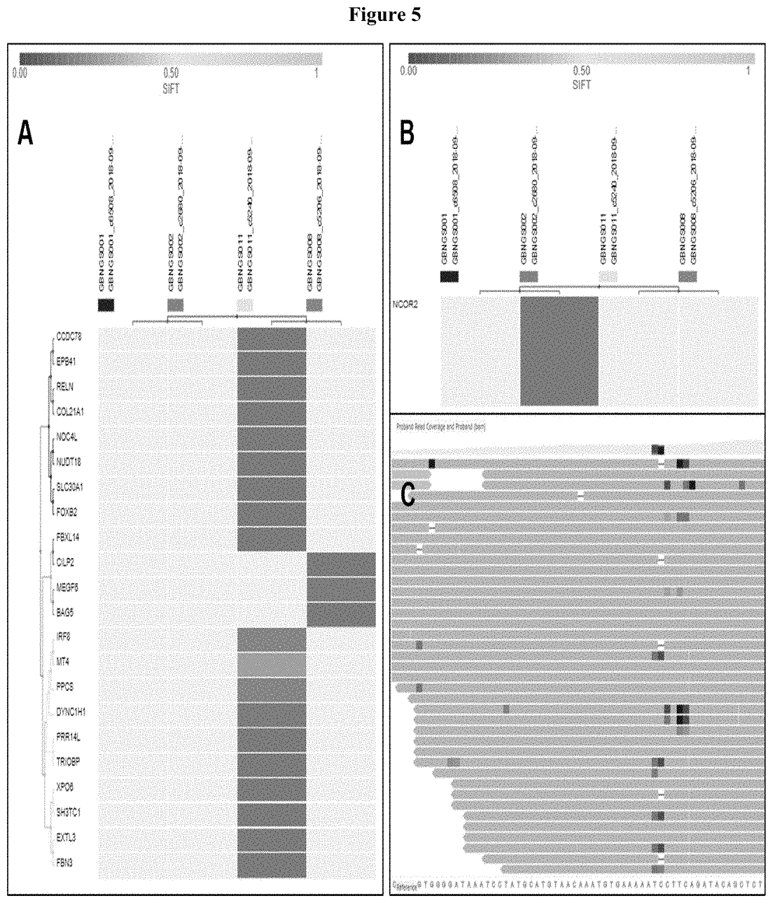

INDEL detection filter chain (FIG. 1D) resulted in 95 variants out of 121,556 associated with 95 involved genes. At first 95 variants were recognized as missense mutations. INDEL-induced frameshift insertion mutation affected 22 genes present in GBNGS008 and GBNGS011. Among reported INDEL-associated frameshift insertion mutation-inflicted genes, three genes (MEGF6, EPB41, PPCS) were found in GBNGS008 and the rest of 17 genes (SLC30A1, SH3TC1, COL21A1, RELN, NUDT18, EXTL3, FOXB2, FBXL14, NOC4L, DYNC1H1, BAG5, CCDCl78, XPO6, MT4, IRF8, FBN3, CILP2) were found in GBNGS011. There was only one GBNGS011 exclusive INDEL-induced nonsense mutation in ZNF333 gene (FIG. 5B). A total of 42 genes were detected with INDEL-associated frameshift deletion mutations. All INDEL-incurring frameshift deletion mutations were found in GBNGS011, except DDX60 in GBNGS001, PIF1 in GBNGS002, and KLHL5 in GBNGS008. Worse functional impact SIFT score ranges from 0.00 represents deleterious effect in genes to 1 represents tolerated effect in genes. Horizontal axis represents gene order distance.

Neoplasm Exclusive Mutations—

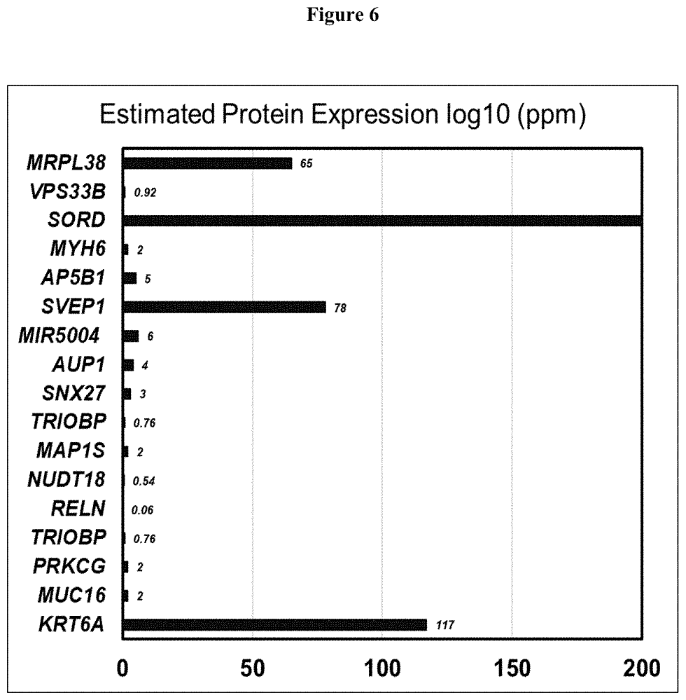

The combination of results from different filter chains revealed neoplasm-exclusive SNV-induced mutation in 31 genes, MNV-induced mutation in 20 genes and INDEL-induced mutation in 5 genes, respectively. Among these candidates, as per “GeneCards” entry 17 genes, viz., KRT6A, MUC16, PRKCG, TRIOBP, RELN, NUDT18, MAP1S, SNX27, AUP1, MIR5004, SVEP1, SORD, VPS33B, MRPL38, AP5B1, and MYH6 showed liver-specific expression (FIG. 6). The ‘x’-axis indicates level of protein expression and ‘y’-axis indicates gene name.

In sum, exome sequencing of four samples was performed to identify critical genetic factor/s associated with liver cancer. By imposing knowledge-based filter chains, a panel of novel genetic variants were revealed. In total, 20 MNV-induced, 5 INDEL-induced, and 31 SNV-induced neoplasm-exclusive genes were revealed through NGS data acquisition followed by data curing with the application of quality filter chains. A liver-specific expression profile of the gene pool identified 17 genes associated with liver cancer. In particular, by sequence analysis, the following 4 novel variants, were identified: c.416T>C (p.Phe139Ser) in SORD, c.1048_1049delGCinsCG (p.Ala350Arg) in KRT6A, c.1159G>T (p.Gly387Cys) in SVEP1, and c.430G>C (p.Gly144Arg) in MRPL38 as critical genetic factor for liver cancer.

### Discussion

In this study, NGS-based exome sequencing of a liver neoplasm patient was preformed against three age-matched asymptomatic subjects where hg19 was used as reference genome for alignment. This experiment resulted a total of 121,556 variants call. A panel of variants for liver cancer was determined through a customized-filter chain of the 121,556 variants. These variants are novel.

A seven-stage filter chain was applied on the entire variant pool. The ultimate target of setting such filtering algorithm was to ensure knowledge-driven variant prioritization exclusive to neoplasm. Two distinct principles were considered for setting the entire filter layer: (1) elimination of population based common variants; and (2) inclusion of functionally significant and unreported cancer variants.

The dbSNP, UCSC common SNPs, DGV and 5000Exomes database were allocated within filter chains for achieving the first goal. The dbSNP and UCSC common SNPs annotation expunged neutral and known phenotypes corresponding to polymorphisms from the variant pool. The DGV hits identified structural variation in the human genome present in healthy samples whereas the 5000Exomes Global MAF is the database of global minor allele frequencies. The cancer risk and treatment outcomes often show population-based variation that is largely attributed to genetic and environmental variation. Therefore, sorting out genetic diversity common to the global population as well as particular ethnic groups was included in the filter chain as exclusive variant prioritization strategies.

After exclusion of possible variants, functional relevance as a second dimension tool was used for identification of non-relevant variant exclusion. The variants were selected through SIFT, Polyphen and Grantham score cutoff, which has been considered associated with worse functional impact on a protein and also damage evolutionary distance. Specific filter chains were applied thereafter for gathering COSMIC unmatched variant to call cancer exclusion variants. A typical WES-data generates large numbers of genetic variants. Prioritization of the variants in the context of disease study incorporates urge of sorting functional relevant variants. Thus, fixing these two filets in the filter chain enabled searching disease-relevant variants.

A pool of 17 genes was selected from liver-specific expression profile. Identified genes were quite diversified in their biological significance and disease association. KRT6A encodes for Keratin 6A and is involved in wound healing; defects in this gene primarily lead to hypertrophic nail dystrophy (Pachyonychia Congenita 3 and Pachyonychia Congenita 1). Cell surface associated Mucin 16 (MUC16) is used as a marker for different cancers and associated with Ovarian Cyst. Protein Kinase C Gamma (PRKCG) is a member of the serine- and threonine-specific protein kinases family that phosphorylates p53/TP53 and promotes p53/TP53-dependent apoptosis in response to DNA damage. TRIOBP encodes for TRIO and F-Actin Binding Protein. By interacting with trio, TRIOBP controls actin cytoskeleton organization, cell motility and cell growth. Reelin, encoded by RELN, regulates cell-cell interactions and modulates cell adhesion. Nudix Hydrolase 18 (NUDT) is linked to purine metabolism. Microtubule-associated protein (MAP1S) mediates mitochondrial aggregation and consequential apoptosis. Sorting Nexin Family Member 27 (SNX27) is involved in recycling of internalized transmembrane proteins. AUP1 encodes for Lipid Droplet Regulating VLDL Assembly Factor, a protein that plays an essential role in the quality control of misfolded proteins in the endoplasmic reticulum and lipid droplet accumulation. MIR5004 is an RNA gene that codes for the miRNA, MicroRNA 5004. This miRNA is affiliated with RET proto-oncogene signaling. SVEP1 encodes “EGF and Pentraxin Domain Containing 1”. SVEP1 is associated with calcium ion binding and chromatin binding. SORD encodes Sorbitol Dehydrogenase and is associated with cataracts and microvascular complications of diabetes 5. MRPL38 encodes for Mitochondrial Ribosomal Protein L38, which plays a role in organelle biogenesis and maintenance and mitochondrial translation. The protein encoded by AP5B1 (Adaptor Related Protein Complex 5 Subunit Beta 1) is involved with Hereditary Spastic Paraplegia. Myosin Heavy Chain 6 (MYH6) is associated with ERK Signaling and cytoskeleton remodeling. Defects in Myosin Heavy Chain 6 causes Atrial Septal Defect 3 and cardiomyopathy.

Among these 17 genes, four genes: MRPL38, SORD, SVEP1 and KRT6A, showed the highest level of expression.

The present methodology which utilizes a number of databases in a filter chain, thus, was useful to identify novel disease-associated variants.

Relevant portions of references referred to herein are incorporated by reference.

1. A method of identifying disease-associated gene variants in a patient comprising:
i) obtaining a nucleic acid sample from a patient and conducting exome sequencing of the sample to identify nucleic acid variants within the sample;
ii) filtering out non-disease-related nucleic acid variants by comparison with known sequence variants from the specific ethnic background, somatic mutations and common non-disease-related sequence variants; and
iii) conducting a comparison of filtered sequence against a healthy control nucleic acid sequence to identify disease-associated sequence variants.

2. A method of identifying a gene variant of at least one of KRT6A, MUC16, PRKCG, TRIOBP, RELN, NUDT18, MAP1S, SNX27, AUP1, MIR5004, SVEP1, SORD, VPS33B, MRPL38, AP5B or MYH6, or protein it encodes, in a patient sample comprising the steps of:
i) contacting a biological sample obtained from the patient with a reactant that binds to at least one of the gene variants or proteins; and
ii) detecting the presence of the gene variant or protein in the sample by detecting binding of the reactant with the gene variant or protein.

3. The method of claim 2, wherein the reactant is a detectably labelled oligonucleotide probe that is complementary to the target gene variant and specifically hybridizes to the target gene variant.

4. The method of claim 2, wherein the reactant is an anti-double stranded DNA (anti-dsDNA) antibody that binds to a target gene variant.

5. The method of claim 2, wherein the reactant is an antibody that binds to a protein encoded by a gene variant.

6. The method of claim 2, wherein the gene variant is a variant of SVEP1, SORD, MRPL38 or KRT6A.

7. The method of claim 2, wherein the gene variant is a variant of SORD comprising a mutation at position 416 in which T is replaced with C.

8. The method of claim 2, wherein the gene variant is a variant of KRT6A in which the GC at position 1048-1049 is replaced by CG.

9. The method of claim 2, wherein the gene variant is a variant of SVEP1 in which the G at position 1159 is replaced with T.

10. The method of claim 2, wherein the gene variant is a variant of MRPL38 in which G at position 430 is replaced with C.

11. The method of claim 2, wherein binding of the reactant to the gene variant or protein it encodes is detected by immunoassay.

12. The method of claim 2, wherein the patient sample is selected from the group consisting of blood, urine, saliva, cerebrospinal fluid or a tissue sample.

13. A method of diagnosing liver cancer in a patient comprising the steps of:
i) contacting a biological sample obtained from the patient with a reactant that binds to at least one gene variant selected from the group of KRT6A, MUC16, PRKCG, TRIOBP, RELN, NUDT18, MAP1S, SNX27, AUP1, MIR5004, SVEP1, SORD, VPS33B, MRPL38, AP5B1, and MYH6, or protein it encodes;
ii) detecting the presence of at least one of the gene variants or proteins it encodes in the sample by detecting binding of the reactant with the gene variant or protein; and
iii) diagnosing the patient with liver cancer when the presence of the gene variant or protein is detected.

14. The method of claim 13, wherein the gene variant is a variant of SVEP1, SORD, MRPL38 or KRT6A.

15. The method of claim 14, wherein the gene variant is a variant of SORD comprising a mutation at position 416 in which T is replaced with C; a variant of KRT6A in which the GC at position 1048-1049 is replaced by CG; a variant of SVEP1 in which the G at position 1159 is replaced with T; or a variant of MRPL38 in which G at position 430 is replaced with C.

16. The method of claim 13, additionally comprising the step of treating the patient with at least one of chemotherapy, radiation or surgery.
